(19) United States
(12) Patent Application Publication
(10) Pub. No.: US 2019/0147988 A1
(43) Pub. Date: May 16, 2019

(54) HOSPITAL MATCHING OF DE-IDENTIFIED HEALTHCARE DATABASES WITHOUT OBVIOUS QUASI-IDENTIFIERS

(71) Applicant: KONINKLIJKE PHILIPS N.V., EINDHOVEN (NL)

(72) Inventors: Reza SHARIFI SEDEH, Malden, MA (US); Daniel Robert ELGORT, New York, NY (US); Roel TRUYEN, Turnhout (BE)

(21) Appl. No.: 16/091,574

(22) PCT Filed: Apr. 19, 2017
(86) PCT No.: PCT/EP2017/059266

§ 371 Date: Oct. 5, 2018

Related U.S. Application Data
(60) Provisional application No. 62/324,363, filed on Apr. 19, 2016.

(51) Int. Cl. G16H 10/60; G06F 16/22; G06F 16/2455

(52) U.S. Cl. CPC G16H 10/60; G06F 16/2455; G06F 16/22

(57) ABSTRACT

An electronic processor (14) is programmed to perform integration (16) of N anonymized healthcare databases (10). For in a pair of databases (i,j) of the N anonymized healthcare databases, a set of features is identified (44) each contained in both databases i and j of the pair of databases (i,j). A conversion table is generated (46, 48) that matches patients of the pair of databases based on patient similarity measured by the set of features. The identifying and generating operations are repeated (50) for each unique pair of databases of the N anonymized healthcare databases to generate N(N 1)/2 conversion tables (20). The electronic processor is further programmed to perform a patient data retrieval process (18) which receives a patient ID of a patient in one of the N anonymized healthcare databases and retrieves patient data for the patient contained in the N anonymized healthcare databases using the N(N−1)/2 conversion tables.

### FIELD

The following relates generally to the medical research and development arts, the healthcare database curation arts, healthcare data mining arts, and related arts.

### BACKGROUND

Numerous areas of healthcare research and development leverage healthcare databases containing data on medical patients. Medical histories or other clinical data, patient billing data, administrative records pertaining to matters such as hospital bed occupancy, and so forth are maintained by hospitals or other medical facilities and/or by individual units such as the cardiac care unit (CCU), intensive care unit (ICU), or emergency admittance department. These databases store sensitive patient data that generally must be maintained confidentially under financial and/or medical privacy laws such as (in the United States) the Health Insurance Portability and Accountability Act (HIPAA).

To enable a patient database to be used for data analytics for clinical, hospital administrative, or other purposes while maintaining patient privacy, it is known to anonymize the database by removing patient-identifying information (PII). Information that needs to be anonymized includes patient name and/or medical identification number (suitably replaced by a randomly assigned number or the like), address, or so forth. Other anonymization measures may include removing “rare” patients who might be identifiable by a combination of unusual characteristic for example, a patient who is 102 years old with a particular illness might be identified on the basis of that information alone.

In addition to rare patients, a patient might be identifiable based on timestamp information for events recorded in the patient record. For example, if a patient is admitted to the hospital on a certain date with a certain condition, that information may be sufficient to narrow the number of possible patient identifications to a small number. However, longitudinal information, that is, the time sequence of events and the time intervals between various events, is sometimes useful in healthcare data analytics. For example, the time interval between admission and discharge may be useful or even critical for analyzing hospital efficiency and/or effectiveness of a certain treatment. To reduce the potential for using a timestamp to identify an anonymized patient while retaining the longitudinal information potentially of value for the healthcare data analysis, in some anonymized databases the timestamps are shifted by some random amount (generally different for each patient), using a rigid shift for all timestamped events of a given patient. The random rigid time shift in timestamps makes patient identification via timestamp more difficult, while the use particularly of a rigid time shift retains the longitudinal information, i.e. the time interval information between events.

### SUMMARY

In one disclosed aspect, an anonymized healthcare data source device comprises at least one electronic processor programmed to integrate N anonymized healthcare databases (10) where N is a positive integer having a value of at least three by performing a database integration process including the operations of: for a pair of databases (i,j) of the N anonymized healthcare databases, identifying a set of features each contained in both databases i and j of the pair of databases (i,j) and generating a conversion table matching patients of the pair of databases based on patient similarity measured by the set of features; repeating the identifying and generating operations for each unique pair of databases of the N anonymized healthcare databases to generate N(N−1)/2 conversion tables. The at least one electronic processor is further programmed to perform a patient data retrieval process including the operation of retrieving patient data for one or more anonymized patients contained in the N anonymized healthcare databases using the N(N−1)/2 conversion tables.

In another disclosed aspect, an anonymized healthcare data source device comprises at least one electronic processor programmed to integrate a healthcare database i and a healthcare database j by performing a database integration process including the operations of: for the pair of databases (i,j), identifying a set of features each contained in both databases i and j of the pair of databases (i,j) including at least one longitudinal feature defined by a pair of timestamped events separated by a time interval Δt between the timestamps of the events and generating a conversion table matching patients of the pair of databases (i,j) based on patient similarity measured by the set of features including comparison of the time interval Δt for patients in the two databases (i,j). The at least one electronic processor is further programmed to perform a patient data retrieval process including the operation of retrieving patient data for one or more anonymized patients contained in both anonymized healthcare databases (i,j) using the conversion table matching patients of the pair of databases (i,j). In another disclosed aspect, a non-transitory storage medium stores instructions readable and executable by a computer to perform an anonymized population image reconstruction method to reconstruct an anonymized population image from N anonymized healthcare databases where N is a positive integer having a value of at least two. The anonymized population image reconstruction method comprises: for a pair of databases (i,j) of the N anonymized healthcare databases, identifying a set of features each contained in both databases i and j of the pair of databases (i,j) and generating a conversion table matching patients of the pair of databases based on patient similarity measured by the set of features. The identifying and generating operations are repeated for each unique pair of databases of the N anonymized healthcare databases to generate the anonymized population image comprising contents of the N anonymized healthcare databases integrated by the N(N−1)/2 conversion tables.

One advantage resides in providing for integration of two, three, four, or more anonymized healthcare databases to leverage the combined data contained in the databases for healthcare data analytic tasks.

Another advantage resides in providing for the foregoing in which one or more anonymized healthcare databases is an unstructured healthcare database.

Another advantage resides in providing the foregoing in which longitudinal information, that is, time intervals between events, is leveraged in matching anonymized patients in different anonymized healthcare databases.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a medical analytics device that leverages anonymized patient data integrated from two or more anonymized healthcare databases.

FIG. 2 diagrammatically illustrates an embodiment of the databases integration process performed by the device of FIG. 1 configured to integrate three or more anonymized healthcare databases.

FIG. 3 shows a table diagrammatically demonstrating criterion for selection of different feature integrating different anonymized healthcare databases.

FIG. 4 diagrammatically shows operation of the refinement component of the databases integration process embodiment of FIG. 2.

FIG. 5 diagrammatically shows an embodiment of the databases integration process of FIG. 1 that leverages longitudinal information.

### DETAILED DESCRIPTION

Numerous challenges are posed in integration of anonymized healthcare databases. The various anonymized healthcare databases may vary significantly in scope, with only a portion of the data overlapping between any two databases. Indeed, this partial overlap is a significant motivating factor in the desire to integrate multiple anonymized healthcare databases to “fill in” information missing in one database with content from another database. For example, as used herein an “anonymized healthcare database” may be (by way of illustration): a medical records database, such as an anonymized database extracted from a comprehensive Electronic Medical Record (EMR) or a domain-specific medical database such as a cardiovascular information system (CVIS) or an intensive care unit (ICU) information system; an anonymized database extracted from a hospital billing department database; an anonymized database extracted from a medical insurance company database; an anonymized database extracted from a hospital admissions departmental database; or so forth. An anonymized database extracted from a CVIS can be expected to contain medical records pertaining to diagnosis and treatment of cardiovascular disease, but may not include information on insurance coverage for those diagnoses/treatments. By contrast, an anonymized database extracted from the hospital billing department can be expected to contain insurance reimbursement information but not medical diagnosis/treatment data. Combining these databases could provide a more holistic image of the patient population; but the limited content overlap between the two databases which provides motivation for the integration also makes such integration challenging.

In various embodiments disclosed herein, these problems are overcome by leveraging the integration of multiple (three or more) healthcare databases. This can provide a greater degree of overlap overall which motivates toward performing integration of the N databases in a single process; however, paradoxically it is disclosed herein that a more efficient and reliable approach for performing the integration is to first integrate each pair of anonymized healthcare databases, so as to generate a conversion table for each pair, and then refine the resulting N(N−1)/2 conversion tables based on consistency of patient matching between the N(N−1)/2 conversion tables. This approach recognizes that the overlap of features between the N databases is likely to be small, and moreover even where overlap is present certain features may be unreliable in some databases. By employing the disclosed approach of first integrating pairs of databases, a set of features can be chosen for each such pairwise integration that is well-chosen for that pair of anonymized healthcare databases. The additional information provided by the multiple (N>2) databases is then leveraged in the subsequent refinement step, which in some embodiments does not rely upon the features.

Additionally or alternatively, in embodiments disclosed herein these problems are overcome by leveraging longitudinal information, that is, the time sequence of events and the time intervals between various events. In general, a longitudinal feature is defined by a pair of timestamped events for a single anonymized patient in an anonymized healthcare database which are separated by a time interval Δt between the timestamps of the events. Such longitudinal features are well-defined even in an anonymized healthcare database in which the anonymization process introduces a random, but rigid, shift of all timestamps for each patient, since the rigid time shift does not affect the time intervals Δt between events.

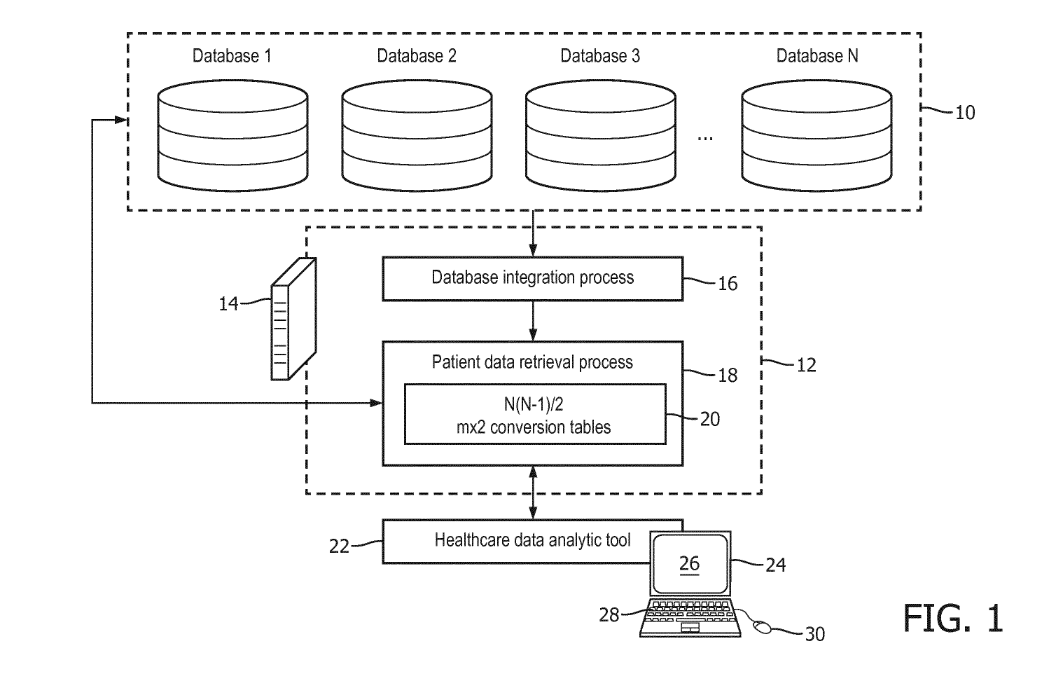

With reference to FIG. 1, N anonymized healthcare databases 10 are denoted “Database 1”, “Database 2”, . . . , “Database N”, respectively. In general, N is a positive integer which is at least two, and in some embodiments is at least three. The lower limit of N=2 is contemplated in some embodiments. The anonymized healthcare databases 10 are generated by a suitable anonymization process (not shown), which are preferably automated (e.g. computer-implemented, with the computers programmed to remove certain classes or types of data) in order to anonymize large databases, e.g. a million patient entries or more in some embodiments. Optionally, the anonymization may also include some manual processing, for example to remove certain rare patients or to address other unusual situations. The anonymization processes employed to generate the N anonymized databases may in general be different, and/or may or may not anonymize the same information. Each anonymization process preferably anonymizes personally identifying information (PII) that can immediately identify a patient, such as patient names, patient addresses, social security numbers, or so forth, as well as information that could potentially be PII in combination with other information, such as hospital name, zip code, or so forth. Where information may be PII in combination with other information, it may be sufficient to anonymize only a portion of the combination. For example, the combination of zip code, gender, and date of birth may be personally identifying but by anonymizing only the zip code information acceptable patient anonymity may be achieved. The anonymization process(es) may optionally also remove rare information that could be identifying for certain patients, such as any age over a certain maximum, e.g. 90 years old, and/or diagnoses that are not among a list of common diagnoses, and/or so forth.

In general, the anonymization of a particular datum can be done by removing the data (redaction) or by replacing the data with a placeholder, the latter being preferable in situations where correlations with that particular type of information are desirably retained, albeit with anonymization. For example, medical care unit (e.g. hospital or care unit) entries may be replaced by placeholders that are internally consistent for the database. These placeholders are internally consistent within a given database, but vary essentially randomly between databases. For example, in Database 1 the hospital “Blackacre General Hospital” may be always replaced by the placeholder, e.g. “8243”, while “Whiteacre Community Medical Center” may be always replaced by the placeholder “1238”. In this example, every instance of medical care unit “Blackacre General Hospital” in Database 1 is replaced by (same) placeholder medical care unit “8243” and every instance of medical care unit “Whiteacre Community Medical Center” in Database 1 is replaced by the (same) placeholder medical care unit “1238”. On the other hand, to continue the example for Database 2, each instance of medical care unit “Blackacre General Hospital” in Database 2 may be replaced by the same placeholder medical care unit “EADF” (which is different from the placeholder “8243” used for Blackacre in anonymized Database 1), and each instance of “Whiteacre Community Medical Center” may be replaced by the same placeholder medical care unit “JSDF” (which again is different from the placeholder “1238” used for Whiteacre in anonymized Database 1). Such anonymization of medical care units by medical care unit placeholders that are internally consistent within the anonymized database enables a healthcare data analytic process operating on a database to identify correlations with a particular medical care unit while maintaining patient anonymity. For example, if Blackacre has a statistically significantly higher success rate for heart transplants than the average hospital, this will show up in Database 1 (assuming it stores heart transplant outcome data) as a statistically significantly higher success rate for heart transplants performed at anonymized hospital “8243”.

On the other hand, some information may be anonymized by redaction, that is, removal. For example, residential address information may be redacted entirely, as this is highly identifying and useful correlations with residential address may not be expected for a typical healthcare data analytic process. In a variant embodiment, if residential address correlations are expected to be a useful input for the healthcare data analytic process, address anonymization may be performed by replacing each residential address by a broader geographical area, e.g. the residential city if this city has a sufficiently large population to assure an acceptable level of anonymity. A residential city or county with sufficiently small population may be redacted entirely to avoid retaining “rare” data that could be personally identifying, or may be replaced by a suitably larger geographical unit such as the residential state.

The anonymized healthcare databases 10 are generally expected to each be formatted in some structured format, for example in a relational database format or other structured database format, as spreadsheets, searchable column-delimited rich text files, or so forth. However, in some embodiments one or more of the databases 10 may be an unstructured database, for example storing written text reports on patients, or may have limited structure, e.g. a structured heading providing information such as patient name and demographic information followed by unstructured text reports. In such a case, natural language processing (NLP) may be employed to extract structured representations of the database contents, such as bag-of-words representations of text documents.

As illustrated in FIG. 1, a medical data analytics device includes an anonymized healthcare data source device 12 implemented on a computer 14 (or, more generally, an electronic processor 14), which may for example be a network-based server computer, a cloud computing resource, a server cluster, or so forth. The computer 14 is programmed to perform a database integration process 16 and a patient data retrieval process 18, the latter making use of a set of N(N−1)/2 conversion tables 20. In illustrative embodiments herein, each conversion table is an m×2 conversion table for a pair of databases of the N databases 10. Without loss of generality, the databases of the pair are denoted as database i and database j, respectively, collectively forming the pair of databases (i,j). Each conversion table is an m×2 table having rows (or, alternatively columns) for m patients matched in the pair of databases (i,j) by the database integration process 16, and two columns (or, alternatively, rows) one listing the anonymized patient ID in anonymized database i and the other listing the anonymized patient ID in anonymized database j. For N=2 there is a single pair of databases (i,j). For N>2 there are N(N−1)/2 unique pairs of databases (i,j). This can be obtained using the combination formula for the number of combinations of k elements taken from a set of n:

$$( \begin{matrix}n \\ k\end{matrix} ) = \frac{n ( n - 1 ) … ( n - k + 1 )}{k ( k - 1 ) … 1}$$

In the present case, k=2 since a pair is being drawn, and the set is the N anonymized healthcare databases 10 so that n=N, so the combination reduces to N(N−1)/2. In general, where N>2 the number of matched patients m may differ for different pairs of databases (i,j), although some overlap of patients between database pairs is expected for useful integration of three or more anonymized healthcare databases.

It is contemplated for the N(N−1)/2 conversion tables 20 to be embodied as a single table, e.g. a concatenation of the N(N−1)/2 tables each of dimension m×2 to form a single m×[N(N−1)] table. In this case it is assumed that all N(N−1)/2 constituent m×2 conversion tables have the same number of matched patients m if this is not the case then padding can be used to account for “missing” anonymized patients, e.g. if patient 49 of Database 1 has no match in Database 3 then the constituent m×2 conversion table for the pair (i,j)=(1,3) is suitably filled in by <null> or zeros or other placeholders.

The computer 14 is also programmed to perform the patient data retrieval process 18 to retrieve anonymized patient data from the N anonymized healthcare databases 10 using the N(N−1)/2 conversion tables 20. For example, a query may be submitted to the patient data retrieval process 18 to acquire the value of a query feature for a given patient identified by an anonymized patient ID used in Database 1. This patient ID can be used directly to retrieve the value of the query feature from Database 1, while for each of Databases j=2, . . . , N the appropriate conversion table for the database pair (1,j) is used to match the patient ID in Database j in order to retrieve the query feature value from Database j.

However, in general the query feature may not be contained in all N databases. If the query feature is contained in only one of the N anonymized healthcare databases then the query feature is retrieved from the (single) anonymized healthcare database containing the query feature. On the other hand, if the query feature is contained in two or more of the N anonymized healthcare databases, then a retrieved value is generated for the query feature from the values of the query feature in the two or more of the N anonymized healthcare databases containing the query feature. This may be done, for example, using a feature accuracy metric for the query feature in the respective anonymized healthcare databases containing the query feature. For example, if the query requests the primary diagnosis for patient 49 and Databases 1, 2, and 3 each contain a primary diagnosis field, then this provides three values for primary diagnosis of patient 49 (after conversion of the anonymized patient ID 49 for the Databases 2 and 3 using appropriate m×2 conversion tables). If Databases 1 and 3 are known to have accuracy rates of 97% for primary diagnosis while Database 2 has a much lower accuracy rate (e.g. 71%) for this feature, then the retrieved value is generated as the primary diagnosis obtained from Databases 1 and 3 which are most likely to be accurate. Where different databases store different values for a given query feature, various approaches can be used to generate the retrieved value, such as taking the value for the database of the N databases 10 having the highest accuracy metric for that feature, or taking the most common value (e.g. if six databases list a value for the feature and five of these agree then the value appearing in five of the six databases may be chosen), or in the case of numerical values taking an average of the values (or of the values in some subset of the databases for which the accuracy metric of that feature is highest, or after removing any identifiable outlier values), or so forth.

The queries received and processed by the patient data retrieval process 18 may vary depending upon the purpose of the query. For example, it may be desired to obtain the primary diagnosis for all male patients in the age range 30-50 years old in this case the query might be formulated as a request for the set of primary diagnoses (with an enumeration for each different diagnosis) after appropriate filtering by age and gender. The query result in this case may be the set of data pairs {(diagnosis,count)} where each element (diagnosis,count) stores a text string indicating the diagnosis and a count of the number of patients (after age/gender filtering) having that diagnosis. If the N databases 10 are relational databases then the patient data retrieval process 18 may be implemented as a Structured Query Language (SQL) query engine that receives SQL queries.

With continuing reference to FIG. 1, the healthcare data analytics device further includes a healthcare data analytics tool 22 implemented on a computer 24 (or, more generally, an electronic processor 24), which may for example be a network-based server computer, a cloud computing resource, a server cluster, a desktop computer (as illustrated), or so forth. The computer 24 includes or is operatively connected with one or more display components/devices 26 and one or more user input components/devices such as an illustrative keyboard 28, a mouse or other pointing device 30, a touch-sensitive overlay of the display 26, and/or so forth. The healthcare data analytics tool 22 performs various healthcare analytics such as (by way of illustrative example): assessing insurance coverage for a certain medical procedure; determining survival rates for a medical procedure; assessing demographic correlations with types of medical care most commonly provided to the patient; or so forth. In a suitable embodiment, a user operates the user input device(s) 28, 30 to configure the type of analytic to be performed; the healthcare data analytics tool 22 retrieves appropriate data from the anonymized databases 10 via the patient data retrieval process 18 of the anonymized healthcare data source device 12 and performs the chosen analytical analyses on that data; and the results are presented on the display component(s) 26 as graphical representations or the like, e.g. plotting insurance coverage for a procedure as a histogram binned by date interval, or as a pie chart showing insurance coverage for a procedure with slices corresponding to different insurance companies; or plotting survival rate as a function of geographical location; et cetera.

The illustrative anonymized healthcare data source device 12 is shown in FIG. 1 as being implemented on the computer 14, while the healthcare data analytic tool 22 is shown in FIG. 1 as being implemented on the different computer 24. However, in other embodiments the anonymized healthcare data source device and the healthcare data analytic tool may be implemented on a single computer. Other hardware segmentation topologies are also contemplated, e.g. the databases integration process 16 and the patient data retrieval process 18 could be implemented on different computers. Furthermore, it will be appreciated that the disclosed functionality of the healthcare data analytics device as described herein may be embodied as a non-transitory storage medium storing instructions that are readable and executable by an electronic processor 14, 24 to perform the disclosed functionality. The non-transitory storage medium may, for example, comprise a hard disk drive or other magnetic storage medium, an optical disk or other optical storage medium, a flash memory, read-only memory (ROM), or other electronic storage medium, various combinations thereof, or so forth.

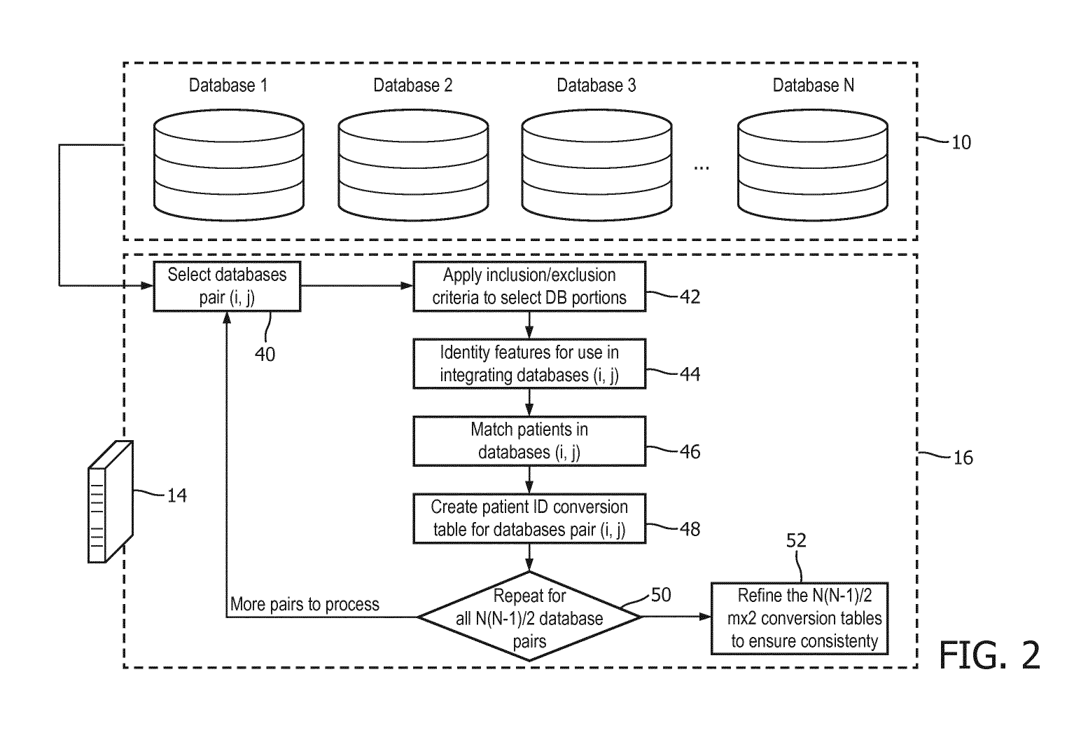

With reference to FIG. 2, an embodiment of the databases integration process 16 for N>2 databases 10 is described. In this embodiment, N is at least three and more generally N could be any positive integer greater than or equal to three. In an operation 40, a (first) pair of anonymized healthcare databases (i,j) are selected from the N databases 10. In one approach, the values of i and j are initially set to one and two, respectively, and will change in each next iteration until all the pairwise combinations of i and j are selected, where 1<i<N and 1<j<N (using the indices 1, . . . , N to denote the constituent databases of the N databases 10. Since the database pair (i,j) integrates two different databases, the pairs exclude all degenerate cases in which i=j.

In the following, an illustrative example is described for matching patients in the chosen databases (i,j). In an operation 42, inclusion/exclusion criteria are applied to select the database portions to match. In order to match the patient-records from Database i and Database j, the subsets of the two databases that are possibly related are extracted. For example, if Database i covers only the data of Medical-surgical and Burn-Trauma ICU patients, from Database j, the subset of patients who were admitted to Medical-surgical and Burn-Trauma ICU wards during their hospitalizations are extracted (i.e. included) while data from other areas that do not overlap Database i are excluded. It should be noted that the excluded/included data is determined by the overlap for the particular database pair (i,j) and may differ for different pairs.

In an operation 44, a set of features is identified for use in integrating the database pair (i,j). Here a set of non-uniquely identifying features is selected with which Database i and Database j can be reliably integrated. The selected features are each contained in both databases i and j of the pair of databases (i,j). Moreover, the selected features are optionally chosen based on available information on reliability. For example, if it is known that one of the databases relatively inaccurate in terms of patients' gender records, but both Database i and Database j are accurate in terms of body weight records, then body weight is suitably chosen as a feature, and gender is suitably not chosen as a feature.

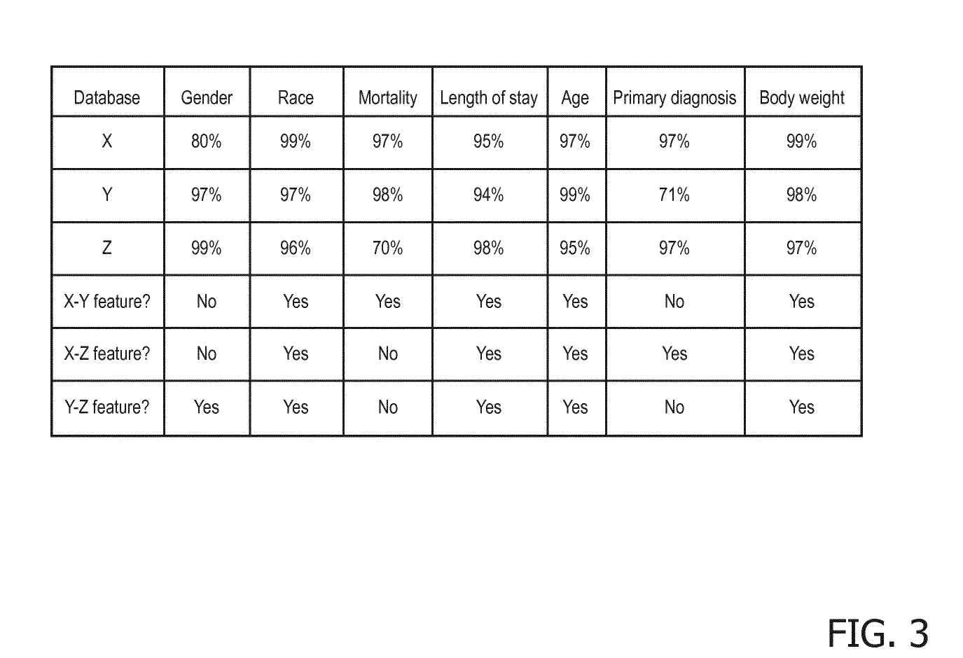

With brief reference to FIG. 3, it is noted that the set of features chosen for integrating a given pair of databases (i,j) depends, in general, on the particular Databases i and j. For example, FIG. 3 shows a table of features for three anonymized healthcare Databases X, Y, and Z, tabulating the accuracy as a percentage for each feature in each database. The last three rows of the table shown in FIG. 3 indicate whether each feature should be selected as the set of features for the indicated database combination i-j. For example, FIG. 3 indicates that Databases X and Y are both accurate in the recording of race, mortality, length of stay, age and body weight, and so these five features are selected for matching Databases X and Y. Likewise, the set of features: race, length of stay, age, primary diagnosis, and body weight is suitably chosen to integrate Database X and Database Z; and the set of features: gender, race, length of stay, age, and body weight is suitably chosen to integrate Database Y and Database Z. In the example of FIG. 3, the accuracy percentages form the feature accuracy metrics, and may be generated based on sampling (selecting a representative sampling of patients and verifying the feature accuracy for the sample), or based on obviously erroneous feature values (e.g. age=0, or age=200), or based on missing feature values (taking each missing feature value as an “error”), or so forth.

With returning reference to FIG. 2, in an operation 46, the set of features chosen in the operation 44 are used to match patients in Databases i and j. Various approaches can be used. In a straightforward approach, a match is found between two patients in Database i and Database j, respectively, if a threshold fraction (or number) of available values for features of the set of features match. Optionally, the matching can apply different weights to the different features based on factors such as the likelihood of having an erroneous recorded feature value in the database, the selectivity of the feature, and so forth. In essence, each patient in Database i is represented by a feature vector whose elements store the values of the set of features selected in operation 44, and likewise each patient in Database j is represented by a feature vector whose elements store the values of the set of features selected in operation 44. Some of these values may be blank (e.g. the vector stores a <null> or other placeholder). Any approach for computing the similarity of two such feature vectors can be used to compare patients and identify similar patients in the two databases. For example, if the number of features is F then a suitable similarity measure may be the distance between the two feature vectors pᵢ and pⱼ given by:

$$D ( {p}_{i} , {p}_{j} ) = \frac{1}{F} {\sum }_{f = 1}^{F} {{w}_{f} ( {p}_{i} ( f ) - {p}_{j} ( f ) )}^{2}$$

where pᵢ and pⱼ are feature vectors representing a patient being compared in Database i and a patient being compared in Database j, respectively, and pᵢ (f) represents the value of the f^{th} feature for patient pᵢ and likewise pⱼ(f) represents the value of the f^{th} feature for patient pⱼ. The parameters w_{f} are feature weights and/or unit conversion factors chosen to indicate the relative importance of the various features f=1, . . . , F and (if necessary) to convert different feature types to a common unit to permit computing the sum. In this formulation, a smaller value for D(pᵢ,pⱼ) indicates more similar patients, so that two patients may be matched if D(pᵢ,pⱼ) is less than some threshold value. Any missing features can be dealt with in various ways, such as simply omitting them from the sum forming D(pᵢ,pⱼ) (and scaling 1/F accordingly), or assigning some default value for pᵢ(f)−pⱼ(f) in the case of a missing feature f. It is to be appreciated that the foregoing is merely an illustrative example and that substantially any other comparison formalism may be used to identify matching patients in the respective Databases i and j.

In an operation 48, the cross-database patient matches identified in the operation 46 are tabulated in a patient ID conversion table for the database pair (i,j). For example, this table may be an m×2 table such as:

TABLE 1
illustrative Patient ID Conversion Table for Database pair (i, j)

| Patient ID (Database i) | Patient ID (Database j) |
|---|---|
| 1 | 43 |
| 2 | 17 |
| 3 | <null> |
| 4 | 98 |
| 5 | 2 |
| 5 | 3 |
| 6 | 6 |

. . .

| | |
|---|---|
| 96 | 9 |
| <null> | 6 |
| <null> | 9 |
| <null> | 23 |

where it will be noted that patient ID=3 in Database i has no match in Database j in this example, and similarly patient ID=6, ID=9, and ID=23 in Database j have no match in Database i. The illustrative example of Table 1 is sorted by patient ID of Database i, but it is trivial to perform a sort by patient ID of Database j if doing so will enable more efficient readout of the table (for example, if the query received by the patient data retrieval process 18 of FIG. 1 is indexed by patient ID in Database j).

It should be noted that in some embodiments the patient matching is not exclusive. This is illustrated in Table 1 where patient ID=5 of Database i is matched with both patient 2 of Database j and with patient 3 of Database j. This optional non-exclusivity enables capture of uncertainties in the patient matching. For medical data analytic applications such non-exclusive matching is not necessarily problematic if the number of such uncertain matches is relatively low, and in such cases allowing for multiple matches in this way can improve the overall accuracy on a statistical basis. In the illustrative conversion table for databases (i,j) shown in Table 1, the storage is by way of duplicate entries for Database i Patient ID 5, which has the advantage of facilitating sorting the table on either the patient IDs of Database i or the patient IDs of Database j.

In a decision operation 50, the processing repeats for each unique pair of databases (i,j) in the set of N databases 10 being integrated, in order to generate a patient ID conversion table for each unique pair of databases (i,j). Thus, this loop will be performed N(N−1)/2 times to generate N(N−1)/2 conversion tables for the N(N−1)/2 unique database pairs obtainable from N databases. For example, if N=3 then there are three iterations, one for the pair (1,2), one for the pair (1,3), and one for the pair (2,3). As another illustrative example, if N=5 then there are ten iterations: (1,2), (1,3), (1,4), (1,5), (2,3), (2,4), (2,5), (3,4), (3,5), (4,5). The loop implemented by decision operation 50 can, for example, be implemented by the nested loop i=1 to N−1; j=i+1 to N (where j is the inner loop).

The output of the N(N−1)/2 loop iterations is the N(N−1)/2 conversion tables for the N(N−1)/2 unique database pairs of the N databases 10. In some embodiments, this is the final output providing the N(N−1)/2 conversion tables 20 (each of dimensions m×2) used by the patient data retrieval process 18. However, if the database integration process 12 terminates at this point then information from the multiple (three or more) healthcare databases (i.e. N>3) is not effectively leveraged to improve the individual m×2 pairwise conversion tables.

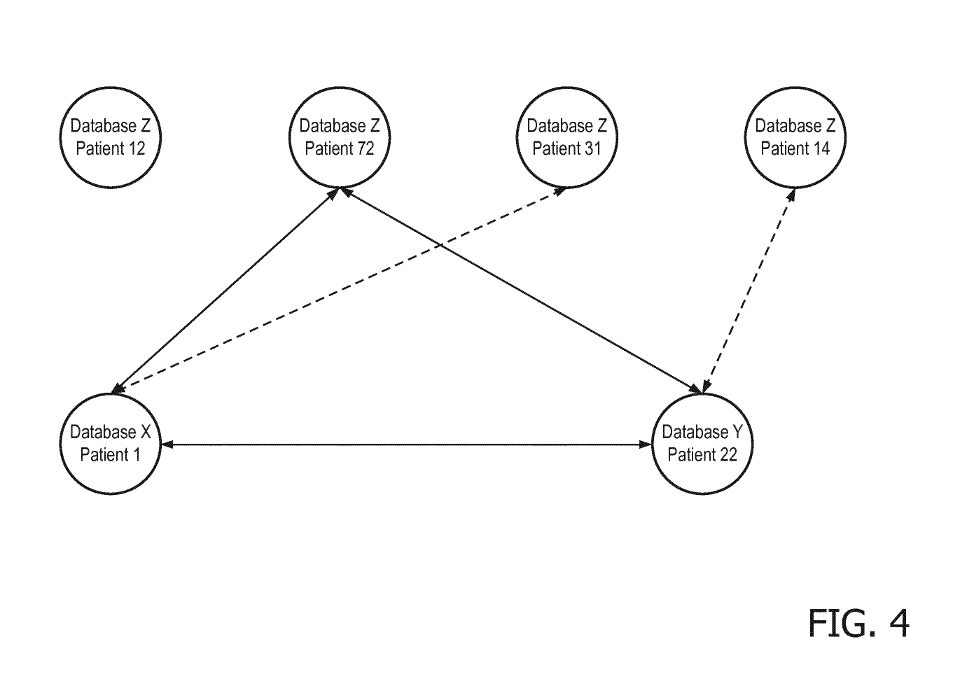

With continuing reference to FIG. 2 and with further reference to FIG. 4, in the illustrative embodiment a refinement operation 52 is performed after the N(N−1)/2 conversion tables are constructed, which refines the N(N−1)/2 conversion tables based on consistency of patient matching between the N(N−1)/2 conversion tables. In an illustrative embodiment, the refinement operation 52 does not use the sets of features identified in the iterations of the operation 44—rather, the refinement operation 52 is performed as diagrammatically shown in FIG. 4, by taking into account the expected consistency between the N(N−1)/2 conversion tables. In the example of FIG. 4, each circle represents a single anonymous patient labeled with his/her anonymized patient ID(e.g. “Patient 1” is labeled with anonymized ID=1) and the database (X, Y, or Z in this example). Solid or dashed lines connecting patients in different databases indicate possible matches found by the pairwise matching of operations 42, 44, 46, 48. In this example, Patient 1 in Database X is linked to Patient 22 in Database Y based on the X-Y conversion table. To maintain consistency, both Patient 1 in Database X and Patient 22 in Database Y should be linked to the same patient in Database Z. However, in the pairwise matching process for the pair i=X, j=Z the Patient 1 of Database X was matched to both Patient 72 and Patient 31 in Database Z (such non-exclusive matching may be permissible as already described for the example of Patient 5 in Database i in the example of Table 1). In the pairwise matching process for the pair i=Y, j=Z the Patient 22 of Database Y was matched to both Patient 72 and Patient 14 in Database Z. To maintain self-consistency it follows that both Patient 1 of Database X and matched Patient 22 of Database Y must match Patient 72 of Database Z the other possible matches are inconsistent. Thus, in the refinement operation 52 the match between Patient 1 of Database X and Patient 31 of Database Z are removed from the X-Z conversion table, and similarly the match between Patient 22 of Database Y and Patient 14 of Database Z are removed from the Y-Z conversion table.

In another embodiment, such consistency analysis could be performed during the iterative loop 40, 42, 44, 46, 48, 50. This approach and reduce processing time for performing later loop iterations by leveraging the already-created pairwise conversion tables. For example, consider the case of N=3 with the databases indexed X, Y, and Z, and with the iterative loop 40, 42, 44, 46, 48, 50 being performed to create the X-Y, X-Z, and Y-Z conversion tables in that order. After creation of the X-Y and X-Z conversion tables it may thereby be known that Patient 10 of Database X is linked to Patient 11 of Database Y, and that Patient 10 of Database X is also linked to Patient 15 of Database Z. Then, during the last iteration to create the Y-Z conversion table, it is already known that Patient 11 of Database Y should be linked to Patient 15 of Database Z in order to assure consistency of the Y-Z conversion table with the already-created X-Y and X-Z conversion tables.

Additionally or alternatively, in some embodiments disclosed herein longitudinal information is leveraged to improve the patient matching. In general, a longitudinal feature is defined by a pair of timestamped events for a single anonymized patient in an anonymized healthcare database which are separated by a time interval Δt between the timestamps of the events. Such longitudinal features are well-defined even in an anonymized healthcare database in which the anonymization process introduces a random, but rigid, shift of all timestamps for each patient, since the rigid time shift does not affect the time intervals Δt between events.

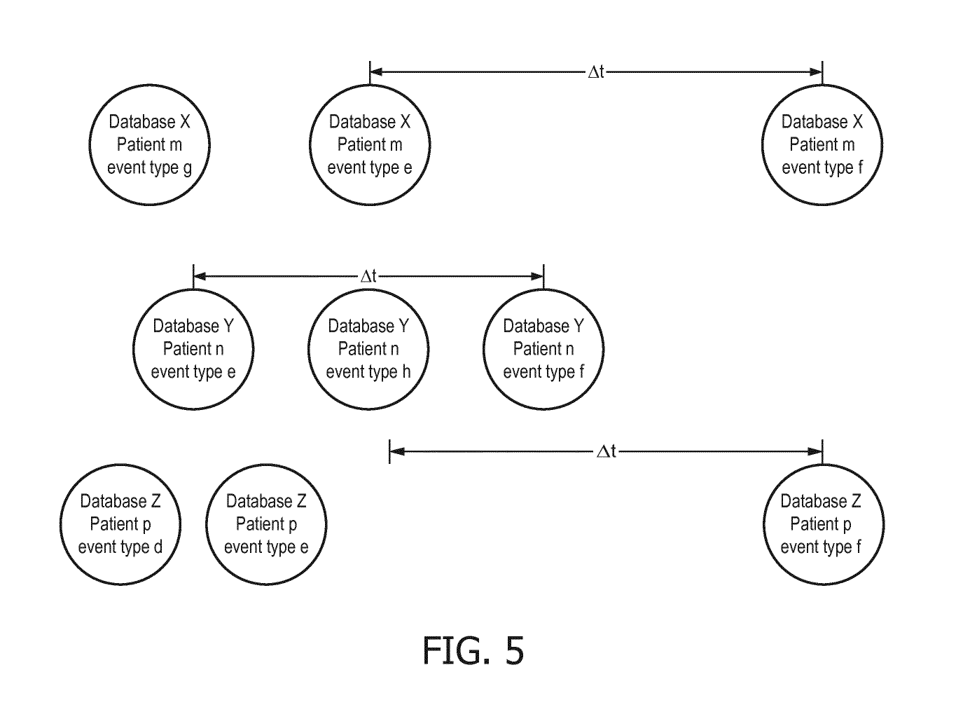

With reference to FIG. 5, an example of a longitudinal feature is described. The longitudinal feature is defined by an event of type e followed by an event of type f which are separated by time interval Δt. In the example of FIG. 5, a Patient m in Database X has an occurrence of an event of event type e followed by an occurrence of an event of event type f which are separated by time interval Δt. Likewise, a Patient n in Database Y has an occurrence of an event of event type e followed by an occurrence of an event of event type f which are separated by the same time interval Δt. By contrast, a Patient p in Database Z has an event of event type e followed by an event of event type f—however, the time interval between the events of types e and f, respectively, is much greater than the time interval Δt. Thus, based on the temporal feature of event sequence e→f separated by time interval Δt, the Patient m in Database X matches the Patient n in Database Y but does not match the patient p in Database Z. In matching such longitudinal features it is contemplated to allow for some variation in Δt for the patients in different databases to account, for example, for possible errors in entry of the timestamps.

It is contemplated to have more complex longitudinal features, e.g. events of types g→e→f with events g e separated by a first time interval Δt₁ and events e→f separated by a second time interval Δtₜ. In other contemplated longitudinal features, the allowable variation in Δt may be large enough that practically the longitudinal feature is matched if the events of types e→f occur in sequence regardless of the time interval between them (within some limit defined by the allowable variation in Δt).

The illustrative longitudinal features employ the time interval Δt between events, rather than comparing timestamps of events for patients in the two databases (i,j). As discussed previously, this approach relying upon time intervals between events, rather than relying on absolute timestamps of events, is robust against the possibility that the patient timeline was rigidly shifted by a random amount as part of the anonymization process.

In some embodiments, the longitudinal features are treated like other features of the set of features identified in operation 44 and used in operation 46 (see FIG. 2). However, this approach may introduce unnecessary computational complexity since identification and processing of longitudinal features can be computationally complex. For example, if the average patient has E=30 events then the number of pairwise event comparisons needed to identify a longitudinal feature of the form e→f is E(E−1)/2=435 event pairs. On the other hand, the rather high specificity of longitudinal features means they can be highly discriminatory for matching patients. Accordingly, in some embodiments the patient matching operation 46 is initially performed without reliance upon longitudinal features, with the longitudinal features being computed and leveraged only for difficult matches (e.g., a patient in Database X that matches more than one patient in Database Y when only the non-longitudinal features are used).

In some embodiments, the non-longitudinal feature matching is performed (or is performed in part) using a universal patient ID(or UID) for each patient. The UID is constructed as a concatenation of a set of common features such as the patient's gender, race, age, and body weight. For example, the UID 1518170 for a patient could be generated using their following features: Male or Gender 1 (the first digit of 1518170); Native American or Race 5 (the second digit of 1518170), Age of 18 years (the third and fourth digits of 1518170) and body weight of 170 pounds (the fifth, sixth, and seventh digits of 1518170). Hence, every time a new record (medical report or claims record) is generated for a patient, a UID is assigned to the patient-record. Since the UID is feature-based, it should be the same across different anonymized databases. Optionally, some tolerance is accepted, e.g. Age of 80 in Database II is considered to be the same as Age of 79-81 in Database I, when using the tolerance threshold of ±1 year for Age. Such a UID approach for feature matching may be employed for all features of the set of features used to match the patient, or alternatively a smaller sub-set of features may be concatenated to form the UID, where the set of features forming the UID are common to all N databases 10. This latter approach advantageously enables the UID to be computed once and re-used for each iteration of the (i,j) loop of FIG. 2, which can increase computational efficiency. In this approach a three-level matching process is contemplated: (1) match based on UID; (2) for difficult cases, match based on additional non-longitudinal features not included in the UID; and finally (3) for even more difficult cases match using longitudinal features.

It will be appreciated that various combinations of disclosed aspects may be employed in a given embodiment. For example, longitudinal feature matching can be used both for dual-database integration (N=2) and for multi-database integration (N≥3). Natural language processing (NLP) can be used to generate a set of features from an unstructured or semi-structured database for both N=2 and N>3 integration tasks.

In an alternative approach for viewing the disclosed healthcare data analytics device of FIG. 1, the process of integrating the N anonymized healthcare databases 10 can be viewed as an anonymized population image reconstruction method to reconstruct an anonymized population image from the N anonymized healthcare databases 10. In this alternative view, the reconstructed anonymized population image comprises contents of the N anonymized healthcare databases 10 integrated by the N(N−1)/2 conversion tables 20. In this alternative viewpoint, the anonymized population image reconstruction method reconstructs (or transforms) population imaging data in the form of the N anonymized healthcare databases 10 into the anonymized population image comprising the contents of the N anonymized healthcare databases 10 integrated by the N(N−1)/2 conversion tables 20.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An anonymized healthcare data source device comprising:
at least one electronic processor programmed to integrate N anonymized healthcare databases where N is a positive integer having a value of at least three by performing a database integration process including the operations of:
for a pair of databases of the N anonymized healthcare databases, identifying a set of features each contained in both databases i and j of the pair of databases and generating a conversion table matching patients of the pair of databases based on patient similarity measured by the set of features;
repeating the identifying and generating operations for each unique pair of databases of the N anonymized healthcare databases to generate N(N−1)/2 conversion tables; and
the at least one electronic processor further programmed to perform a patient data retrieval process including the operation of retrieving patient data for one or more anonymized patients contained in the N anonymized healthcare databases using the N(N−1)/2 conversion tables.

2. The device of claim 1 wherein identifying the set of features for the pair of databases includes identifying features for which a feature accuracy metric satisfies a minimum accuracy for each anonymized healthcare database of the pair of databases.

3. The device of claim 1 wherein retrieving the patient data contained in the N anonymized healthcare databases includes, for a query feature:
if the query feature is contained in only one of the N anonymized healthcare databases then retrieving the query feature from the anonymized healthcare database containing the query feature; and
if the query feature is contained in two or more of the N anonymized healthcare databases then generating a retrieved value for the query feature from the values of the query feature in the two or more of the N anonymized healthcare databases containing the query feature based on the feature accuracy metric for the query feature in the respective anonymized healthcare databases containing the query feature.

4. The device of claim 1 wherein generating the conversion table includes generating an m×2 conversion table where m is the number of patients matched in the pair of databases.

5. The device of claim 1 wherein the database integration process includes the further operation of refining the N(N−1)/2 conversion tables based on consistency of patient matching between the N(N−1)/2 conversion tables.

6. The device of claim 5 wherein the refining does not use the identified sets of features.

7. The device of claim 1 wherein the database integration process includes, for at least one pair of databases of the N anonymized healthcare databases:
identifying at least one longitudinal feature defined by a pair of timestamped events separated by a time interval Δt between the timestamps of the events; and
generating the conversion table matching patients of the pair of databases based in part on matching of the longitudinal feature including comparison of the time interval Δt for patients in the two databases.

8. The device of claim 7 wherein generating the conversion table matching patients of the pair of databases based in part on matching of the longitudinal feature does not include comparison of timestamps of events for patients in the two databases.

9. An anonymized healthcare data source device comprising:
at least one electronic processor programmed to integrate a healthcare database i and a healthcare database j by performing a database integration process including the operations of:
for the pair of databases, identifying a set of features each contained in both databases i and j of the pair of databases including at least one longitudinal feature defined by a pair of timestamped events separated by a time interval Δt between the timestamps of the events and generating a conversion table matching patients of the pair of databases based on patient similarity measured by the set of features including comparison of the time interval Δt for patients in the two databases;
the at least one electronic processor further programmed to perform a patient data retrieval process including the operation of retrieving patient data for one or more anonymized patients contained in both anonymized healthcare databases using the conversion table matching patients of the pair of databases.

10. The device of claim 9 wherein generating the conversion table matching patients of the pair of databases based on patient similarity does not include comparison of timestamps of events for patients in the two databases.

11. The device of claim 9 wherein:
identifying the set of features includes identifying a set of non-longitudinal features contained in both databases i and j of the pair of databases and, for each patient in each database i and j, generating a universal identifier (UID) for the patient comprising a concatenation of values of the set of non-longitudinal features for the patient; and
generating the conversion table includes generating the conversion table matching patients of the pair of databases based on patient similarity measured by the set of features further including comparison of the UIDs for patients in the two databases.

12. The device of claim 9 wherein:
identifying the set of features includes identifying at least one feature in at least one database of the pair of databases by performing natural language processing (NLP) on text content of patient records to extract the feature.

13. The device of claim 9 wherein identifying the set of features each contained in both databases i and j of the pair of databases includes identifying features for which a feature accuracy metric satisfies a minimum accuracy for both the anonymized healthcare database i and the anonymized healthcare database j.

14. The device of claim 9 wherein retrieving the patient data contained in both anonymized healthcare databases using the conversion table matching patients of the pair of databases includes, for a query feature:
if the query feature is contained in only one database of the pair of anonymized healthcare databases then retrieving the query feature from the anonymized healthcare database containing the query feature; and
if the query feature is contained in both databases of the pair of anonymized healthcare databases then generating a retrieved value for the query feature from the values of the query feature in the pair of anonymized healthcare databases based on the feature accuracy metric for the query feature in the respective anonymized healthcare databases containing the query feature.

15. The device of claim 9 wherein generating the conversion table includes generating an m×2 conversion table where m is the number of patients matched in the pair of databases.

16. The device of claim 9 wherein:
the at least one electronic processor is programmed to integrate N databases including the anonymized healthcare database i, the anonymized healthcare database j, and at least one additional anonymized healthcare database by performing the database integration process including the further operation of repeating the identifying and generating operations for each unique pair of databases of the N anonymized healthcare databases to generate N(N−1)/2 conversion tables; and
the at least one electronic processor is further programmed to perform the patient data retrieval process including the operations of receiving a patient ID of a patient in one of the anonymized healthcare databases and retrieving patient data for the patient contained in the N anonymized healthcare databases using the N(N−1)/2 conversion tables.

17. A non-transitory storage medium storing instructions readable and executable by a computer to perform an anonymized population image reconstruction method to reconstruct an anonymized population image from N anonymized healthcare databases where N is a positive integer having a value of at least two, the anonymized population image reconstruction method comprising:
for a pair of databases of the N anonymized healthcare databases, identifying a set of features each contained in both databases i and j of the pair of databases and generating a conversion table matching patients of the pair of databases based on patient similarity measured by the set of features; and
repeating the identifying and generating operations for each unique pair of databases of the N anonymized healthcare databases to generate the anonymized population image comprising contents of the N anonymized healthcare databases integrated by the N(N−1)/2 conversion tables.

18. The non-transitory storage medium of claim 17 wherein the stored instructions are readable and executable by a computer to further perform an anonymized population image data retrieval method including receiving an anonymized population data query and retrieving patient data responsive to the anonymized population data query from the anonymized population image using the N(N−1)/2 conversion tables.

19. The non-transitory storage medium of claim 17 wherein N is a positive integer having a value of at least three.

20. The non-transitory storage medium of claim 19 wherein generating the conversion table includes generating an m×2 conversion table where m is the number of patients matched in the pair of databases whereby each of the N(N−1)/2 conversion tables is an m×2 conversion table.

21. (canceled)

22. (canceled)

23. (canceled)

24. (canceled)
